(19) United States
(12) Patent Application Publication
(10) Pub. No.: US 2011/0264022 A1
(43) Pub. Date: Oct. 27, 2011

(54) ORTHOPEDIC CAST TAPE SYSTEM

(75) Inventors: Jeffrey L. Freeman, Burlington, NC (US); Walter D. Doubleday, Jupiter, FL (US)

(73) Assignee: CAROLINA NARROW FABRIC COMPANY, Winston-Salem, NC (US)

(21) Appl. No.: 12/767,891

(22) Filed: Apr. 27, 2010

(51) Int. Cl. A61F 5/00

(52) U.S. Cl. 602/6

(57) ABSTRACT

An orthopedic casting system that includes a cast tape having a moisture responsive resin therein, the tape being wound onto a tubular polymeric core for shipment and storage prior to use in forming an orthopedic cast. The tubular core has a wall with an inner and outer surface in a first position comprising a plurality of relatively shallow convex and concave portions that extend linearly of the core. Each of the concave portions provide a longitudinal trough along which water flows adjacent the inner wraps of the cast tape. The wall of core is flexibly resilient so that the upon application of a radial inward force on the cast tape and the core, the wall will flex to a second position in which the troughs are deepened. When the force is released the tubular core returns to substantially the first position.

### FIELD OF THE INVENTION

The present invention is directed to an orthopedic cast tape system, and in particular to a uniquely configured core for use with orthopedic cast tapes that improves the distribution of moisture to the cast tape wrapped around the core.

### BACKGROUND

Orthopedic cast tapes with moisture responsive resins conventionally are subjected to water to cure the resin in the tape and form the cast to treat wrist, arm, ankle and leg fractures, as well as muscle strains. Cast tapes have been supplied on cores with an outer packaging used to keep moisture away from the resin impregnated on the cast tape until time for use. Typically, a doctor, nurse, orthopedic technician or other person removes the cast tape from the packaging and, prior to application to the patient, introduces moisture to the resin by exposure to ambient conditions, or by submerging the cast tape still on the core in water. Either method has its drawbacks, in that sufficient moisture may not adequately be exposed to the maximum amount of resin prior to the onset of cure. The patient's limb is then wrapped with the tape which is then allowed to cure. This in turn may cause an irregular, or even in some cases, an ineffective cure of the tape to form the cast.

### SUMMARY OF INVENTION

The inventor has addressed the problem of irregular and inefficient cure of orthopedic casts by developing an improved orthopedic cast system, including a uniquely configured tubular core. An aspect of the invention is an orthopedic casting system with a cast tape having a moisture responsive resin therein and a tubular core. The cast tape is wound onto a tubular core for shipment and storage prior to use in forming an orthopedic cast. The tubular core is formed of a flexible polymeric material and includes a wall with an inner and outer surface. The core wall has a plurality of relatively shallow convex and concave portions that extend linearly of the core. When the cast tape is wound on a core, the concave portions provide a series of longitudinal troughs along which water flows adjacent the inner wraps of the cast tape. Further, the wall of the tubular core is flexibly resilient so that upon application of a radial inward force on the cast tape and the core, the wall will flex to a second position in which the troughs become deepened, allowing even freer flow of moisture. When the force is released, the tubular core returns to substantially its initial position. The plurality of convex and concave portions and the flexible resilience of the wall improve the distribution of moisture along the core and into the cast tape.

Another aspect of the invention is the tubular core itself upon which an orthopedic cast tape is wound. The core is formed of a tubular flexible polymeric material and has a wall with an inner and outer surface. The core wall has a plurality of relatively shallow convex and concave portions that extend linearly of the core. Again when the cast tape is wound on a core, the concave portions provide a longitudinal trough along which water or moisture flows adjacent the inner wraps of the cast tape. Further, the tubular wall of the core is flexibly resilient so that upon application of a radial inward force on the cast tape and the core, the wall will flex to a second position in which the troughs are deepened. When the force is released, the tubular core returns to substantially the first position.

### BRIEF DESCRIPTION OF THE SEVERAL VIEWS OF THE DRAWINGS

FIG. 1 is a perspective view of an orthopedic cast system without the packaging thereon.

FIG. 2 is a front end view of the orthopedic cast system without the packaging thereon.

FIG. 3 is a perspective view of a tubular core without the orthopedic cast tape wrapped thereon.

FIGS. 4A and 4B are schematic views of the end of a tubular core of the orthopedic cast system.

### DETAILED DESCRIPTION OF THE INVENTION

Certain exemplary embodiments of the present invention are described below and illustrated in the accompanying figures. The embodiments described are only for purposes of illustrating the present invention and should not be interpreted as limiting the scope of the invention, which, of course, is limited only by the claims below. Other embodiments of the invention, and certain modifications and improvements of the described embodiments, will occur to those skilled in the art, and all such alternate embodiments, modifications, and improvements are within the scope of the present invention.

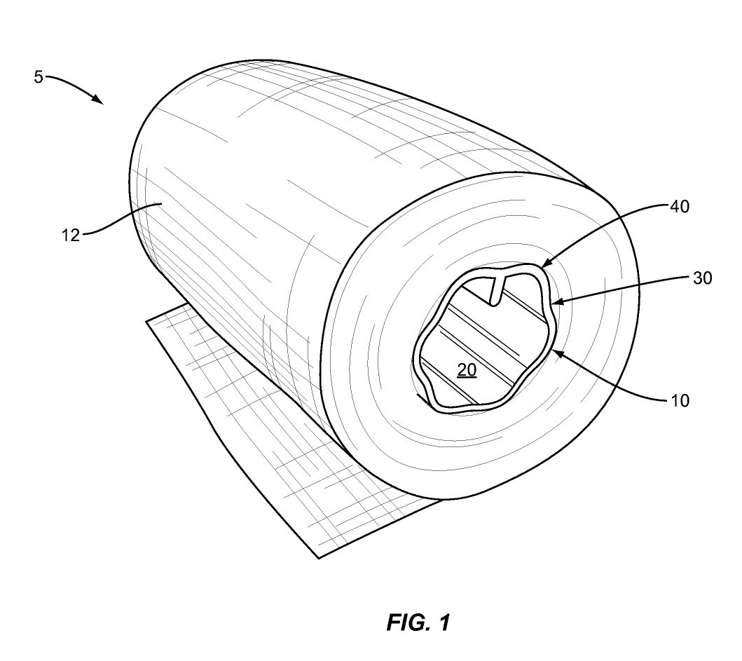

FIG. 1 shows an improved orthopedic casting system 5 that includes a cast tape 12 with a moisture responsive resin therein, and a tubular core 10 on which the cast tape 12 is wrapped. The casting system 5 may be stored and shipped in a sealed package (not shown) to prevent the exposure of the cast tape resin to moisture which might otherwise initiate a premature cure of the cast tape.

The improved orthopedic casting system 5 may be used by an orthopedic technician, doctor, nurse or other person to treat a broken bone or muscle strain as described above. In use, the casting system 5 is first removed from the package. The core 10 with the cast tape 12 thereon is then exposed to moisture or submerged in water. The user applies some radial force, or flexes, the core 10 to help facilitate movement of the water along the core 10 and into the cast tape 12 as will be discussed in more detail below. When the adequate amount of moisture has been exposed to the cast tape and resin, the cast tape 12 is removed from the core 10 and wrapped around the wound site. The cast tape will then set or cure through the normal curing process.

In exemplary embodiments, the cast tape 12 may be between 3 to 6 inches wide. The length of the core 10 would approximate the width of the cast tape 12. However, the core 10 can be any length and can correspond to any width cast tape 12 as is needed for the particular wound or injury.

The cast tapes themselves may be woven or warp knit fabrics. The fabrics may be formed from fiber that include, but are not limited to, glass, polyester, polypropylene, aramid, and polyamides. The cast tape includes a moisture curable resin impregnated thereon. For example, a conventional polyurethane-based resin is an exemplary type of resin used.

The tubular core 10 is formed primarily of a polymeric material and may be extruded to an indeterminate length and cut to the requisite size for the particular orthopedic cast system to be used. The core 10 may be formed from a group of polymers selected from the group consisting of linear low density polyethylene (LLDPE), low density polyethylene (LDPE), polyethylene (PE), linear high density polyethylene (LHDPE), high density polyethylene (HDPE), and polyester. Although other polymers may be used as needed.

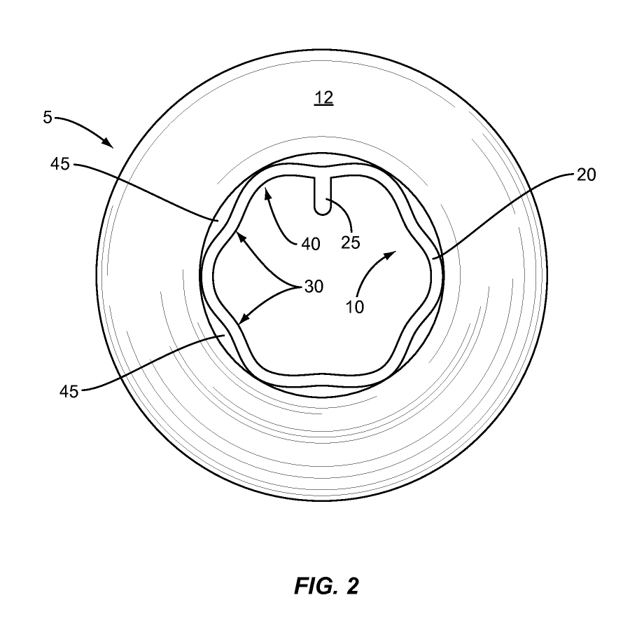

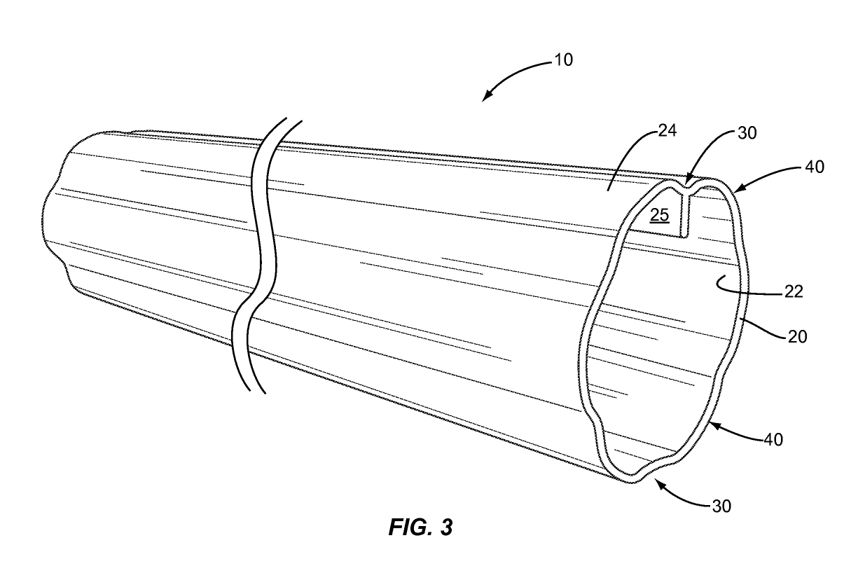

As shown in FIGS. 2 and 3, the core includes a tab 25 extending inwardly from the inner surface 22 of the core 10. The tab 25 helps wrap the cast tape 12 on the core. For example, winding machines typically include a mandrel for receiving a core 10 (without cast tape). Such mandrels include a slot that extends longitudinally along its length. The core 10 slides longitudinally onto the surface of the mandrel while the tab 25 is received by the slot. One end of the cast tape 12 is positioned on the core 10, and as the mandrel rotates, the core 10 is driven to wrap a length of cast tape thereon. In other alternate embodiments, however, other mechanisms for securing the core onto a mandrel during winding may be used. For example, the core 10 may not have any tab 25. In such a situation, the mandrel may be expansible to engage the inner surface of the core.

Referring to FIGS. 2, 3 and 4A and 4B, the wall 20 includes a plurality of convex portions 40 and concave portions 30 that form longitudinal troughs 45 extending linearly down the length of the core 10. The troughs 45 form an open space between the concave portions 30 of the wall 20 and the inner wraps of the cast tape 12. The troughs 45 allow water to more easily pass down the length of the core 10 when the cast system 5 is submerged in water. In the embodiment shown, the core 10 has between four (4) and six (6) convex portions and between four (4) and six (6) concave portions. In an exemplary embodiment, the core 10 has six (6) convex portions and six (6) concave portions.

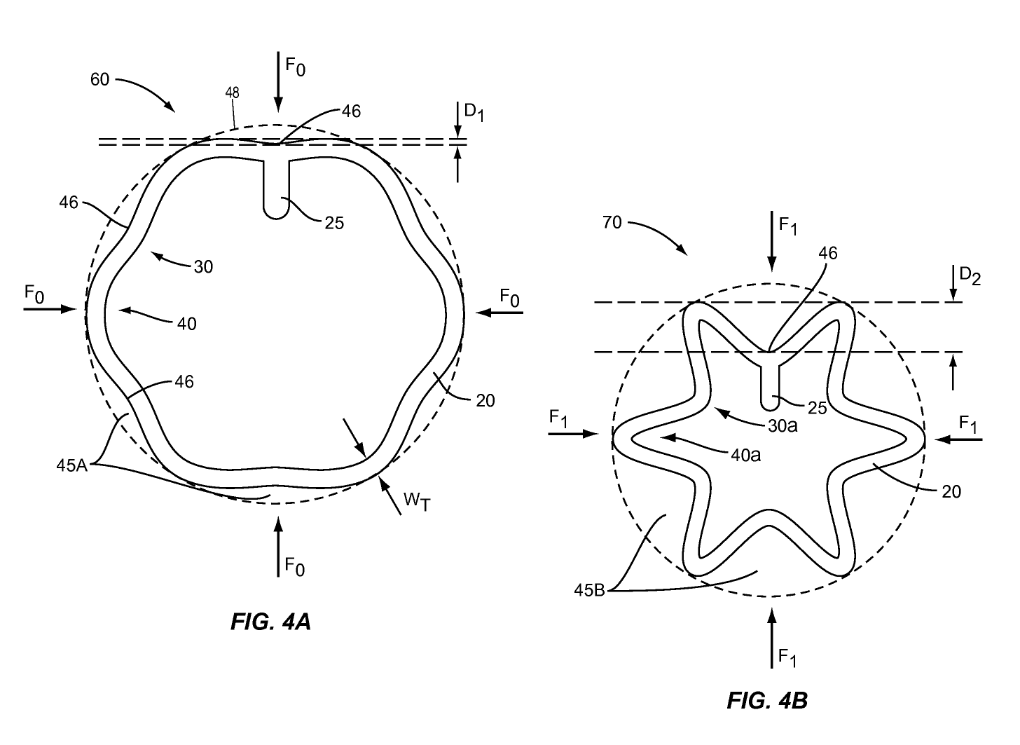

As suggested above, the core 10 may have any geometry that forms the longitudinal troughs 45 discussed, while also providing for a flexibly resilient wall 20. Referring again to FIG. 4A, the wall 20 includes a plurality of points 46 in the bottom of troughs 45 between adjacent convex portions 40. The distance between two parallel lines that are tangent to the perimeter 48 of the circle subtended by the core peaks 40 and point 46 in the relaxed position, respectively, is referred to as the depth or amplitude, D₁. In an embodiment, the amplitude (D₁) is no less than about 0.10 inches (0.254 cm). In an exemplary embodiment, the distance D₁ is 0.12 inches (0.304 cm). A larger amplitude (D₁) corresponds to deeper troughs 45 on the core and as needed, the amplitude (D₁) may be larger or smaller.

An aspect of the core 10 is the ability to distribute water to the cast tape 12. The relatively shallow troughs characterized by the combination of peak-to-peak distance between adjacent convex portions and the amplitude or depth of the concave portions increase the extent of water surface area to cast tape contact. This improves the distribution of water to the resin and promotes a better cure of the cast tape on the wound site. In a preferred embodiment, the peak-to-peak distance may be about 0.486 inches (1.236 cm) for a 0.93 inches (2.36 cm) diameter core. The peak-to-peak distance, however, may be about 0.25 inches or more. As described above, the amplitude D₁ is preferably greater than about 0.10 inches (0.254 cm). In a preferred embodiment, the amplitude D₁ is preferably about 0.12 inches when in the first, un-flexed position. While this particular trough profile has been found to improve water distribution along the core, the number of convex and concave portions also improve water distribution. In a preferred embodiment, a core having between 4 and 6 convex portions yield the desired moisture distribution to the cast tape.

Further, the thickness of wall 20 may influence the flexibility of core 10. Referring to FIG. 4A, the thickness of wall (W_{T}) may range between about 0.02 inches and about 0.06 inches. Preferably, the wall thickness should be no less than about 0.02 inches (0.05 cm). In an exemplary embodiment, the wall thickness (W_{T}) is about 0.04 inches (0.1 cm).

As described above, an aspect of the invention is the flexible resilience of the core 10 that allows the core to move repeatedly between first and second positions. In the embodiment shown in FIGS. 4A and 4B, the core 10 may flex between a first position 60 shown in FIG. 4A to second position 70 shown in FIG. 4B. For illustrative purposes, the view shown in FIG. 4B is an exaggerated view of the second position 70. The application of a force (F₁) to the cast tape 12 and core 10 deepens the longitudinal troughs 45. For example, in FIG. 4A, the wall 20 of the core in its first relaxed position 60 has a first amplitude D₁ and accordingly, a first trough 45A. As shown in FIG. 4B, application of a radially-inward force (F₁) on the cast system 5 flexes the wall 20 into a second position 70 that yields the plurality of convex and concave portions with a second, larger, amplitude, D₂. This flexing has the effect of forming deeper troughs 45B. When the force (F₁) is removed, the core 10 flexes back to substantially the first position 60 having troughs 45A. This action will also force the resin into the inner layers of the wound tape.

When the cast tape 12 and core 10 are flexed while submerged in water, the deepened troughs 45B will facilitate migration of water between the core 10 and the cast tape 12. This improves the likelihood that water will distribute throughout the cast tape more effectively prior to resin cure and setting of the cast tape on the patient.

Although the present invention has been described with exemplary embodiments, it is to be understood that modifications and variations may be utilized without departing from the spirit and scope of the invention, as those skilled in the art will readily understand. Such modifications and variations are considered to be within the purview and scope of the appended claims and their equivalents

What is claimed is:

1. An orthopedic casting tape system, the system comprising;
a cast tape having a moisture responsive resin therein and being wound onto a tubular core for shipment and storage prior to use in forming an orthopedic cast;
the tubular core being formed of a polymeric material and having a flexible wall with an inner and outer surface and in a first position the wall comprising a plurality of relatively shallow, alternating convex and concave portions that extend linearly of the core, the outer surface of the core receiving the cast tape thereon, each of the concave portions providing a longitudinal trough along which, when water is applied to the tape, the water flows adjacent the inner wraps of the cast tape,
the tubular core having between four and six convex portions and four and six concave portions,
the wall having a thickness that is between about 0.02 to about 0.06 inches yet being flexible to the extent that upon application of a radial inward force on the cast tape and the core, the core will flex to a second position in which the troughs are deepened and when the force is released the tubular core returns to substantially the first position;
the tubular core being formed from the group of materials consisting of linear low density polyethylene, low density polyethylene, polyethylene, linear high density polyethylene, high density polyethylene, and polyester; and
an outer package enclosing the cast tape and the core;
wherein the number of convex and concave portions and the flexible resilience thereof improves the distribution of moisture along the core and into the cast tape.

2. The orthopedic casting system of claim 1, wherein the core further comprising a means on the inner surface of the core for securing the core on a mandrel for winding the cast tape thereon.

3. The orthopedic casting system of claim 1, wherein the core further comprises a tab extending inwardly from the inner surface of the core.

4. The orthopedic casting system of claim 1, wherein the core comprises six convex portions and six concave portions.

5. The orthopedic casting system of claim 1, wherein the plurality of relatively shallow convex portions and the plurality of concave portions have an amplitude that is greater than about 0.10 inches.

6. A tubular core upon which an orthopedic cast tape is wound for shipment and storage prior to use in forming an orthopedic cast, the core comprising:
a tubular member having a flexible wall with an inner and outer surface and in a first position comprising a plurality of relatively shallow alternating convex and concave portions that extend linearly of the core, the outer surface of the core receiving the cast tape thereon, each of the concave portions providing a longitudinal trough along which, when water is applied to the tape, the water flows adjacent the inner wraps of the cast tape,
the tubular member further comprises between four and six convex portions and between four and six concave portions,
the tubular member formed from the group of materials consisting of linear low density polyethylene, low density polyethylene, polyethylene, linear high density polyethylene, high density polyethylene, and polyester; and
the wall having a thickness that is between about 0.02 to about 0.06 inches and being flexible to the extent upon application of a radial inward force on the cast tape and the core, the wall will flex to a second position in which the troughs are deepened and when the force is released the tubular core returns to substantially the first position;
wherein the plurality of convex and concave portions and the flexible resilience thereof improve the distribution of moisture along the core and into the cast tape.

7. The orthopedic casting system of claim 6, wherein the core further comprising a means on the inner surface of the core for securing the core on a mandrel for winding the cast tape thereon.

8. The orthopedic casting system of claim 6, wherein the core further comprises a tab extending inwardly from the inner surface of the core.

9. The orthopedic casting system of claim 6, wherein the core comprises six convex portions and six concave portions.

10. The orthopedic casting system of claim 6, wherein the plurality of relatively shallow convex portions and the plurality of concave portions have an amplitude that is greater than about 0.10 inches.
